(19) United States
(12) Patent Application Publication
(10) Pub. No.: US 2004/0033562 A1
(43) Pub. Date: Feb. 19, 2004

(54) Integration of high cell density bioreactor operation with ultra fast on-line downstream processing

(76) Inventor: Alain Miller, Mons (BE)

Correspondence Address: MERCHANT & GOULD PC, P.O. BOX 2903, MINNEAPOLIS, MN, 55402-0903, US

(21) Appl. No.: 10/333,210

(22) PCT Filed: Jul. 24, 2001
(86) PCT No.: PCT/BE01/00121

(30) Foreign Application Priority Data
Jul. 25, 2000 (EP) 00870169.0

(51) Int. Cl. C12P 21/06; C07K 1/14

(52) U.S. Cl. 435/69.1; 530/412

(57) ABSTRACT

The present invention is related to a recovery and purification method of (a) biological(s) of interest produced by cells retained in a bioreactor under appropriate conditions and by appropriate means for producing the biological(s) inside the bioreactor having a volume higher than 1,5 l culture, and cultivated at a high cell density, preferably at a cell density higher than 10×10⁶ cells/ml, said bioreactor being submitted to an acoustic sonoperfusion allowing the recovery of a sonoperfused medium from said bioreactor, characterised in that it is submitted thereafter directly to an Expanded Bed Specific Adsorption for the direct recovery and uninterrupted purification of said biological(s). The present invention is also related to a process unit for recovering and purifying said biological(s) of interest.

### FIELD OF THE INVENTION

The present invention is related to a process unit based upon the integration of a production of cells grown at high density in a bioreactor with a first step of purification scheme (together referred to as the process) for obtaining and purifying biological products such as (recombinant) (glyco/lipo)proteins, peptides, nucleotides and other ‘biologicals’ synthesised from said cells growing in said bioreactor.

The present invention is also related to a method for obtaining and purifying said biological(s) from said cells growing in said bioreactor.

### BACKGROUND OF THE PRESENT INVENTION

Traditionally, growing bacteria or other living cells in the laboratory helps to make biotech products (‘biologicals’) like active pharmaceutical compounds, nucleic acids, amino acids, vitamins, vaccines, enzymes, membranes, receptors, etc.

Today specific genes of interest are isolated and inserted into living organisms whether pro- or eukaryotic (e.g., plant or animal) cells, which will produce them in large amounts, for instance for the production of new vaccines.

Several problems however hamper the scaling-up from laboratory- to industrial-scale of production of high value quality recombinant biologicals.

Biotechnologists must first have the correct cells and the suitable ingredients to feed them. A system (a bioreactor) has to be designed and built for these cells that can be fed and grown successfully under germ-free conditions. The optimum conditions must be determined to stimulate the cells to produce the maximum amounts of the required quality products as to separate, concentrate and purify(operations commonly referred to as to downstream processing) the sought after products.

However, ‘biologicals’ especially the ones obtained from mammalian cells cultivated using industrial bioprocesses in operation today, are tremendously expensive to produce. This results from several limitations: low specific productivity and low cell concentration (2 to 3×10⁶ cells/ml), use of large culture volume (1000 to 5000 l), large-scale and tedious multi-step downstream processing, costly validated spaces, frequent tear downs (long immobilisation periods), etc.

In addition, prolonged exposure of the highly labile ‘biological’ to adverse chemical (proteases, pH, etc.) and physical (temperature) conditions during the downstream processing (very large cell culture medium, long time of incubation between successive batch processes, etc.) has also a strong impact on their final quality and costs.

On the other hand, high cell densities (25 to 50×10⁶ cells/ml) suitable for the production of recombinant molecules at pilot scale (higher than 1,5 l, preferably higher than 5 l culture) have already been proposed. The cells are retained inside the bioreactor by using acoustic sonoperfusion, allowing therefore the desired biological product to be continuously secreted in the sonoperfusate (U.S. Pat. No. 5,527,460 and U.S. Pat. No. 5,519,051). This system allows the cultivation of cells (bacteria, yeast, animal or plant cells), whether anchorage-dependent or not, at high cell concentration in continuously stirred tank reactor (the configuration most favoured by the biotech industry).

Reduction of fixed costs, increased titers and improved quality (shorter residence time in the bioreactor at 37° C.) of the sought after glycoproteins explain the progressive generalisation of this technology.

The document U.S. Pat. No. 5,711,888 describes a multilayer piezoelectric resonator for the separation of suspended particles. In particular, particles suspended in a fluid can be separated and recycled by means of ultrasonic resonating waves. Preferably, the resonating acoustic field is generated within a multilayer composite resonator system including a transducer, the suspension and a mirror parallel to each other.

Such acoustic resonating-wave-based process is suitable for the separation of all kinds of particles (solid, liquid or gaseous dispersed phases) and is applied for the separation of biological particles such as mammalian (whether anchorage-dependent or not), bacterial and plant cells or aggregates therefrom.

This method includes the use of an acoustic filter to achieve the retention of mammalian cells (whether anchorage-dependent or not) in the bioreactor and/or the selective retention of viable cells relative to non-viable ones.

Until now, prior to downstream processing, the high value/quality biological(s)contained in the feedstock must be freed from the secreting living organisms. This is accomplished by either centrifugation or filtration, methods which are complicated to implement, are time-consuming, expensive to run and introduce a discontinuity in the process between cell growth on the one hand and downstream processing on the other.

The European patent application EP-0302724 describes that particulate material can be supported in a fluid medium by means of an ultrasonic standing wave, while a reaction is effected or controlled involving the material so supported, for example with the fluid medium or other material contained in the medium.

### AIMS OF THE INVENTION

The present invention aims to provide a method and process unit that allow to automate and accelerate the recovery and the purification of ‘biologicals’ obtained from cells, preferably mammalian cells, grown at high cell density in a sonoperfused continuously stirred tank reactor (bioreactor), especially high value ‘biological’ such as membranes, (glyco)proteins or peptides suitable for pharmaceutical, medical or other biochemical processes.

A preferred aim of the present invention is to provide such method and process unit which do not present the drawbacks of the methods and process units of the state of the art and which allow the uninterrupted recovery of ‘biological(s)’ in high yield, quality and purity.

A further aim of the present invention is to provide such method and process unit which allow the recovery of biological materials at low cost on an industrial scale.

### SUMMARY OF THE INVENTION

The present invention is related to a process unit combining the technical and economical advantages of a mammalian cell-based bioprocess integrating a high cell density (about 10, but preferably higher than 25×10⁶ cells/ml) reactor coupled to an ultrafast (online) downstream purification processing.

In the process unit and method according to the invention, the cells producing the ‘biologicals’ of interest are retained inside the bioreactor by using an acoustic sonoperfusion system allowing the desired biologicals to be continuously produced in the sonoperfusate, these molecules being thereafter recovered and uninterruptedly purified by direct downstream first purification step.

In order to drastically reduce the “biological's” possible chemical (pH, proteolysis, etc.) and physical (temperature) degradation, the sonoperfusate which is almost completely depleted from cells is advantageously directly and uninterruptedly (i.e., without centrifugation and/or filtration) captured on an Expanded Bed Specific Adsorption (EBSA) chromatography column, the support of which is covalently bound to a ligand specifically adsorbing the sought after ‘biological’ (see FIG. 1).

Therefore, a first aspect of the present invention is related to a recovery and purification method of one or more ‘biological’ of interest produced by cells, preferably mammalian cells (including hybridomas), wherein said cells are retained, preferably in suspension, in a bioreactor by appropriate means for producing the biological product(s) inside the bioreactor having preferably a volume of culture higher than 1,5 l, preferably higher than 5 l culture, at a cell density higher than 10×10⁶ cells/ml.

According to the invention, said bioreactor is submitted to an acoustic sonoperfusion (preferably according to the method described in the patent U.S. Pat. No. 5,711,888, incorporated herein by reference), allowing the recovery of a sonoperfusate (harvest medium) from said bioreactor, being submitted thereafter directly to an Expanded Bed Specific Adsorption (EBSA) (preferably upon materials (chromatographic column) described in the patent application WO99/51316, incorporated herein by reference), for the direct recovery and purification of said ‘biological(s)’, at a purity and recovery rate higher than 75%, preferably higher than 80 or 85%, more preferably higher than 90 or 95% (in weight).

More preferably the Expanded Bed Absorption is performed upon a chromatographic column comprising a dense solid support such as a mineral oxide matrix having a pore volume which is less than 50% of the total volume of the mineral oxide matrix. An interactive polymer network which is rooted in pore and on the surface of the matrix in that said support comprises a specific ligand of the product will be recovered and purified.

It is meant by “(a) biological(s) of interest”, any macromolecule or group of macromolecules of pharmaceutical, medical, or biochemical interest, produced by cell(s), such as viruses, receptors, vaccines, enzymes, nucleic acids or membranes, made of proteinic, glycoproteinic, peptidic, polysaccharidic and/or lipidic structure, including antibodies and hypervariable portions thereof.

Preferably, said biological products are recombinant molecules obtained from recombinant microorganisms, preferably from recombinant mammalian cells (whether anchorage-dependent or not) or hybridomas.

It is meant by “the recovery and the purification of (a) biological(s)”, the specific isolation (on an industrial scale) of said biological(s) from possible living or soluble contaminants such as cells or cell debris.

It is meant by an “uninterrupted” purification and direct recovery upon an Expanded Bed Specific Adsorption, the fact that in the process unit and in the method according to the invention, the sonoperfusate exiting from the bioreactor does not undergo nor a centrifugation nor a filtration before being fed to the EBSA column for the recovery and the purification of the biological(s) of interest.

It is also possible to combine with the method according to the invention, other purification steps for obtaining the biological product of interest highly pure in liquid or solid form.

The present invention is also related to a process unit and a plant comprising several process units with the means for performing the method according to the invention, especially a bioreactor (higher than 1,5 l culture bioreactor), comprising means for integrating a high cell density (higher than 10×10⁶ cells/ml, preferably higher than 25×10⁶ cells/ml) combined with an acoustic sonoperfusion system and means for the recovering of the sonoperfused medium (harvest medium) from said bioreactor, coupled directly to an Expanded Bed Specific Adsorption means (chromatography column) for the recovery and the purification of ‘biological(s)’ of interest produced by said cells and present in said medium.

It is meant by a process unit the combination of several devices which allow preferably continuously the culture of cells and the downstream purification of products obtained from said cells which can be thereafter directly used for their therapeutic or biochemical properties in a highly pure form.

It is meant by a plant comprising several process units the possibility to comprise in a single operation system or factory several of said process units working preferably in parallel for the simultaneous production of various identical or different biologicals according to the GMP requirements.

In the method and process unit according to the invention, the Expanded Bed Specific Adsorption chromatography column comprises specific ligands resistant to the activity of proteases, which are possibly present in the medium.

Advantageously, said chromatography column is an affinity chromatographic column comprising macroporous microbeads made of a matrix such as zircon oxide or quartz characterised by a high porosity, a high physical rigidity, a high specific gravity (with a final density higher than 1.4 g/cm³), an important flow rate (in the range of 500 to 1000 cm³/h), a high mass transfer, a strong chemical stability, able to resist to sanitisation for 2 hours with at least 0.5N NaOH and a high specific binding capacity. Said specific ligands coupled to said beads, are resistant to the activity of possible proteases or other enzymatic molecules present in the sonoperfused medium and, are preferably selected by combinatorial or aptamer chemistry.

The combination of a bioreactor with uninterrupted purification allows advantageously the production of biologicals of interest in high yield and purity and the industrial automation of the production and purification of these high value biologicals.

### SHORT DESCRIPTION OF THE DRAWINGS

FIG. 1 represents an approach for the production and purification of recombinant molecules according to the invention.

FIG. 2 represents the long term (4 weeks) sonoperfused culture of recombinant CHO-K1 cells secreting a recombinant truncated gD protein of an Herpes virus HSV-2 such as described in the patent EP 0 139 417 B2.

FIG. 3a and 3b represent analyses confirming the stability of recombinant protein gD isolated during the plateau cultivation at 25×10⁶ cells/ml (day 0, day 5, day 10, day 15, day 20, day 25 and day 30) (SDS-PAGE/Coomassie staining and lectin analysis).

FIG. 4 represents a SDS-PAGE analysis of monoclonal antibodies recovered by the method according to the invention.

FIG. 5a-5c represent a preferred downstream purification process and system in the method according to the invention.

### DETAILED DESCRIPTION OF THE INVENTION

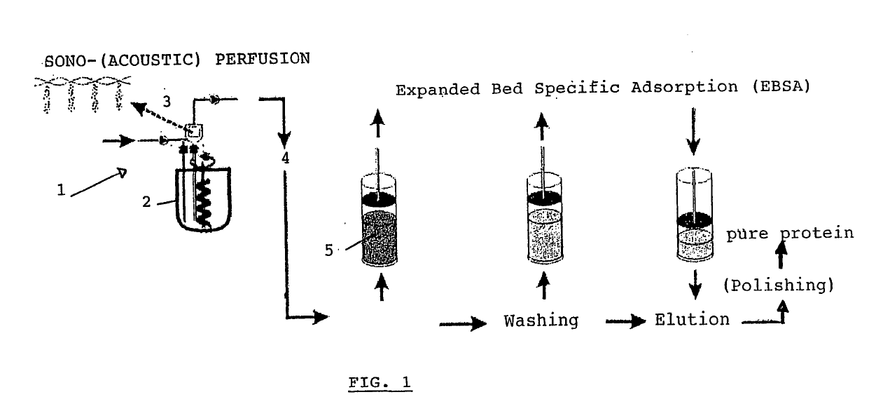

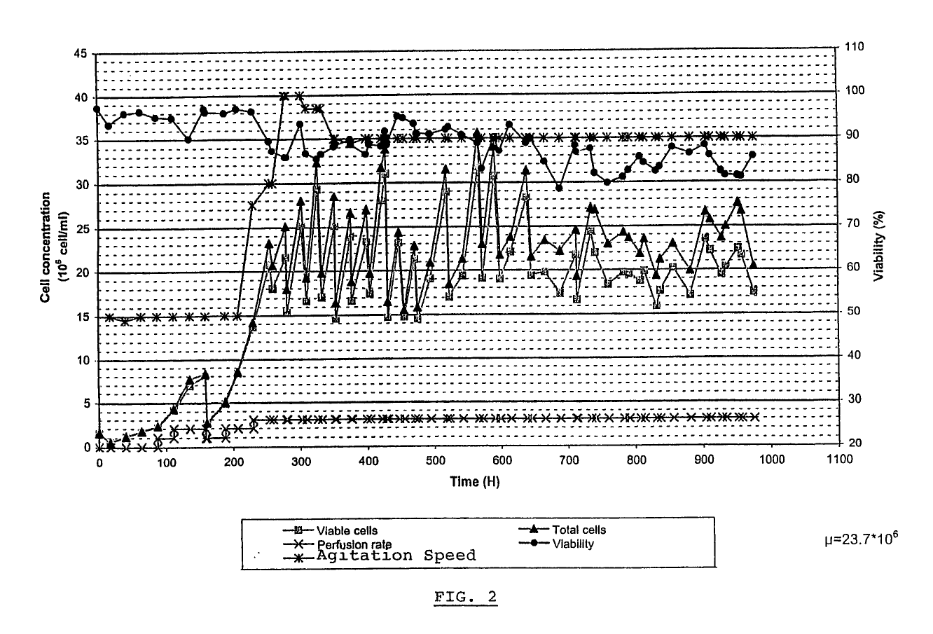

As shown in FIG. 1, the almost quantitative retention of the cells inside a bioreactor 2 of the process unit 1 according to the invention is obtained by acoustic filtration means 3 that allow the recovery of a resulting almost cell-free perfusate (harvest medium) 4 that could be submitted directly in a straightforward way to fractionation by chromatography by using Expanded Bed Specific Adsorption means 5 with zircon oxide or other high dense support.

By the method and process unit according to the invention, it is possible to obtain the recovery of biological molecules such as (glyco/lipo)proteins with a high purity.

With the present invention, pitfalls resulting from the method of the state of the art are completely eliminated, giving rise to a process which integrates in a single operation cell growth and downstream plant or process unit, a direct and single-step clarification, concentration and initial fractionation at a high yield and purity, this at low costs.

As expected, the biologicals of interest (recovered and purified) maintain their integrity and can be used for their advantageous therapeutic or industrial properties.

Furthermore, by using specific ligands in the zircon oxide-based Expanded Bed Specific Adsorption according to the invention, it is possible to solve the drawbacks of the state of the art such as degradation due to the presence of proteases in the harvest medium.

The present invention will be described in more details in the following non-limiting examples.

### EXAMPLE 1

The process unit according to the invention comprises a 8 l culture volume bioreactor and two acoustic sorting flow cells working synergistically (obtained from Anton Paar GmbH, Austria) for the growth of a CHO-K1 cell line. The 8-l culture volume bioreactor is adapted for high density cell culture in sonoperfusion (up to 25×10⁶ cells/ml). Said cell concentration could be maintained during a long period (for at least four weeks).

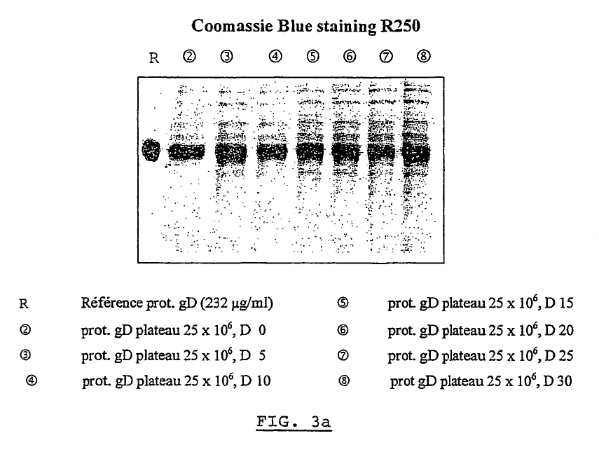

The CHO K1 cell line was selected for its ability to produce a recombinant truncated gD protein of an Herpes virus HSV-2 such as described in the patent EP 0 139 417 B2. The recombinant protein produced at a high cell concentration (15 to 33×10⁶ cells/ml) exhibits the same biochemical characteristics as a reference expressed and purified by classical techniques (see FIG. 3a and 3b, and table 2).

The harvest obtained from the bioreactor according to the invention was submitted to an Expanded Bed Specific Adsorption purification made out of a column with zircon oxide beads bound to a ligand selected from an industrial textile dye library. Said ligand has been immobilised upon the agarose or another gel incorporated in said beads.

Taking into account binding and elution capacity (elution yield and protein gD purity in eluates), the specific dye according to the invention has been selected as the most suitable specific dye for the protein gD from cell culture contaminants (cells and cell debris, proteins secreted in the medium, etc.).

Other ligands, especially other dyes, could be selected by the person skilled in the art according to routine experiments based upon the affinity constant and the capacity value of each ligand to recover the biological molecule of interest.

The various parameters and the characteristics for the recovering of proteins are presented in the following table 1.

TABLE 1

| | |
|---|---|
| Volume of perfusate | 40 l |
| Volume of packed specific | 0.2 l |
| ligand bound zircon oxide | |
| particles | |
| Volume of elution (3 × the | 0.6 l |
| volume of packed specific | |
| ligand bound support) | |
| Total capacity | 4.6 mg recombinant protein/ |
| | ml specific ligand bound on |
| | zircon oxide support |
| Loading time | 6 l/h |
| Eluted recombinant protein | ≧85% (SDS-PAGE/ |
| purity | 1-D densitometry) |
| Eluted recombinant protein | ≧85% (ELISA) |
| recovery | |

The characteristics of said protein (molecular weight, 3D conformation, chromatogram, etc.) have shown that the recovered glycoprotein has maintained its integrity, in the recovery and purification method and plant (process unit) according to the invention.

Quality control of protein gD produced and purified during the whole cultivation process (4 weeks) has been confirmed by studying the glycosylation status of samples isolated during the plateau cultivation at 25×10⁶ cells/ml (day 0, day 5, day 10, day 15, day 20, day 25 and day 30).

TABLE 2

| Time of | | GD | |
|---|---|---|---|
| plateau | prot tot | protection | specific |
| cultivation | harvest | harvest | productivity |
| (D = day) | (μg/ml) | (μg/ml) | μg/10⁶ cell |
| D 0 | 217 | 48 | 6.6 |
| D5 | 280 | 60 | 6.8 |
| D10 | 253 | 35 | 5.2 |
| D15 | 262 | 52 | 6.5 |
| D20 | 292 | 48 | 5.3 |
| D25 | 282 | 37 | 4.9 |
| D30 | 316 | 55 | 8.0 |
| Average | 272 | 48 | 6 |
| Standard | 31 | 9 | 1 |
| dev. | | | |

These purified samples have confirmed a totally similar glycosylation status by their monosaccharide composition, identical lectin detection (see FIG. 3b), and by the characterisation of the N-glycan chains (MALDI-TOF-MS).

### EXAMPLE 2

Production of monoclonal antibodies (IgG) secreted from a hybridoma cell line cultivated at high cell density and purified by EBSA (Expanded Bed Specific Adsorption) with the r-ProteinA (recombinant protein A) Streamline® gel.

A murine hybridoma cell line was grown in DMEM growth medium supplemented with 10% Foetal Calf Serum (FCS).

Hybridoma cells for the Master Cell Bank were grown in Techne glass spinner vessels until a 160-ml volume at a concentration of 1.6×10⁶ cells/ml, providing 25 cryotubes of 10⁷ cells/tube.

The Working Cell Bank (WCB) was established from fresh stocks of hybridoma cells thawn from an ampoule, the cells being grown afterwards in DMEM growth medium supplemented with 10% FCS.

It was initially decided to progressively increase the cell concentration (at an 1.5-1 scale) until 2.5×10⁷ cells/ml and, in a second phase, to maintain this cell concentration during a long period (10 days). The concentration of the secreted IgG was determined by using a specific mouse IgG ELISA assay (Roche No1333151, Mannheim, Germany)

In a preliminary step, the binding and elution conditions were optimised by using a column specifically dedicated to analytical experiments on fluidised bed (FastLine 10, Upfront, DK).

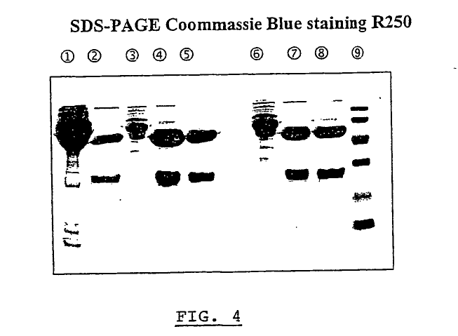

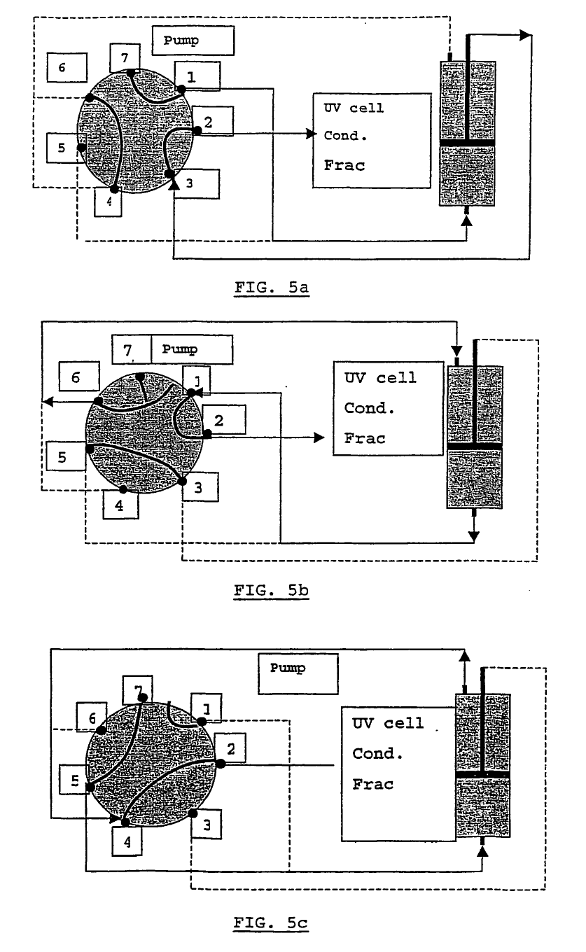

6-litre samples of a 2.5×10⁷ cells/ml culture sonoperfusate were collected each day to perform the downstream purification process which was fully automated by using the Akta Prime purification system (Amersham-Pharmacia 18-1137-18, Sweden; see FIG. 5a-5c, where FIG. 5a=load position, FIG. 5b=injection position and FIG. 5c=waste treatment position). The monoclonal antibody was captured in an expanded mode using a S25 column (Amersham Pharmacia, 18-1110-50) and 100 ml of the Streamline® rProteinA resin (Amersham Pharmacia, 17-1281-01). The gel was thereafter washed with a Phosphate Buffer saline (PBS) at pH 7.5 in an expanded and packed mode successively (200 ml and 75 ml, respectively). The IgG was eluted in a packed mode (75 ml) at pH 4.5 (elution buffer: 50 mM sodium acetate, pH 4.5).

The eluted IgG protein was thereafter characterised and analysed by SDS-PAGE densitometry in order to determine its purity. The antibody reactivity was estimated by Western blot analysis and quantified by ELISA.

Cultivation at 2.5×10⁷cells/ml was maintained at this cell density for 10 days without experiencing a dramatic decrease in cell viability (>80%; see table III). By using a 1.5 l bioreactor, the specific productivity averaged 4.3 μg IgG/10⁶ cells/ml/day allowing a total production higher than 142 mg of antibody per day.

The monoclonal antibody was directly captured from unclarified and undiluted sonoperfused culture medium without adjustments (neutral pH, independence of ionic strength).

After an extensive wash with PBS, the isocratic elution of the antibody was obtained under mild acidic conditions at low ionic strength (elution buffer: 50 mM sodium acetate, pH 4.5).

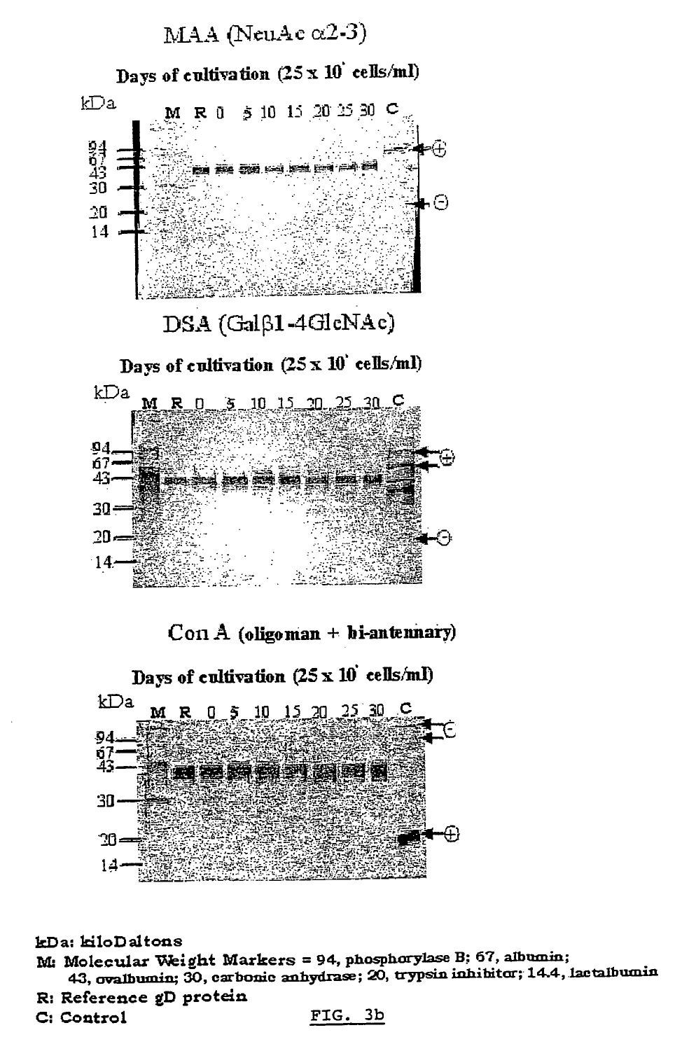

For five different experiments, SDS-PAGE (Coommassie staining) and Western blot analyses confirmed a protein purity higher than 99% and an excellent reactivity against the target antigen (see FIG. 4). The rProtein A ligand with a capacity above 10 mg protein IgG/ml resin can be considered as an excellent candidate for the capture of this IgG from the sonoperfusate.

SDS-PAGE, Western blot, ELISA and Gel filtration analyses performed in parallel unambiguously showed that no apparent difference are detected for the antibody binding capacity and purity.

Five 6-litre batches of hybridoma culture medium (grown at 2.5×10⁷ cells/ml) were purified. Western blot and ELISA analyses clearly indicated that the IgG was completely captured from 6 litres of sonoperfusate (more than 100 mg of IgG) with a purity higher than 99%.

\mathring{1} Harvest—Day 01

\mathring{2} Purified IgG—Batch 01

\mathring{3} Supernatant—Day 02

\mathring{4} Purified IgG—Batch 02,

\mathring{5} Purified IgG—Batch 03,

\mathring{6} Supernatant—Day 03,

\mathring{7} Purified IgG—Batch 04,

\mathring{8} Purified IgG—Batch 05,

\mathring{9} Low Molecular Weight markers: 94; 67; 43; 30; 20.1 and 14.4 kDa

TABLE III
Culture Parameters (one run)

| | | | Specific |
|---|---|---|---|
| Time of | | | productivity |
| plateau | Total Cell | | (μg lgG/10⁶ |
| cultivation | density | Viability | cells/ |
| (day) | 10⁶ cells/ml) | (%) | ml/Day) |
| D0 | 18 | 86 | 5 |
| D1 | 24 | 77 | 6.3 |
| D2 | 25 | 71 | 4.9 |
| D3 | 21 | 74 | 5 |
| D4 | 22 | 78 | 4 |
| D5 | 24 | 79 | 2.8 |
| D6 | 25 | 79 | 3.8 |
| D7 | 22 | 79 | 3.1 |
| D8 | 26 | 80 | 2.1 |
| D9 | 21 | 82 | 2.8 |
| D10 | 18 | 81 | 3.6 |
| Average | 22 | 80 | 4.3 |

TABLE IV
Purification table

| | | | | Purified | | |
|---|---|---|---|---|---|---|
| | Supernatant | [IgG] | Quantity | IgG | Recovery | Purity |
| Exp. | volume (L) | (mg/L) | (mg) | (mg) | (%) | (%) |
| 01 | 6 | 27 | 162 | 143 | 88 | >99 |
| 02 | 5.8 | 37 | 215 | 186 | 86 | >99 |
| 03 | 5.9 | 31 | 183 | 165 | 90 | >99 |
| 04 | 6.1 | 31 | 189 | 180 | 95 | >99 |
| 05 | 5.7 | 25 | 142 | 125 | 88 | >99 |

1. Recovery and purification method of a biological of interest produced by cells retained in a bioreactor under appropriate conditions and by appropriate means for producing the biological(s) inside the bioreactor having a volume higher than 1,5 l culture at a high cell density, preferably at a cell density higher than 10×10⁶ cells/ml, said bioreactor being submitted to an acoustic sonoperfusion allowing the recovery of a sonoperfused medium from said bioreactor, characterised in that it is submitted thereafter directly to an Expanded Bed Specific Adsorption for the direct recovery and purification of said biological.

2. Method according to the claim 1, characterised in that the cells are mammalian cells (whether anchorage-dependent or not), including hybridomas.

3. Method according to the claim 1 or 2, characterised in that the biological is a macromolecule or a group of macromolecules being an active pharmaceutical compound or an enzyme.

4. Method according to any one of the preceding claims, characterised in that the Expanded Bed Specific Adsorption is performed upon a chromatography column comprising dense solid support, such as a mineral oxide matrix, having a pore volume which is less than 50% of the total volume of the mineral oxide matrix, and an interactive polymer network which is rooted in pores and on the surface of the matrix and in that said support comprises specific ligands to the biological(s) of interest to be recovered and purified.

5. Method according to the claim 4, characterised in that the ligand is protease-resistant and specific to the biological of interest to be purified and recovered.

6. Process unit (1) for the recovery and the purification of a biological of interest, characterised in that it comprises a bioreactor (2) having a volume higher than 1,5 l culture comprising means for integrating a high cell density, preferably a density higher than 10×10⁶ cells/ml, inside the bioreactor, combined with an acoustic sonoperfusion system and means (3) for the recovery of a sonoperfused medium (4) from said bioreactor (2), coupled directly to an Expanded Bed Specific Adsorption means (5) for the recovery and the purification of the biological(s) of interest produced by said cells and present in said medium.

7. Process unit according to the claim 6, characterised in that the cells are mammalian cells (whether anchorage-dependent or not) including hybridomas.

8. Process unit according to the claim 6, characterised in that the cells are other eukaryotic cells (yeast, insect cells, etc.) or prokaryotic cells (bacteria, etc.).

9. Process unit according to the claims 6 to 8, characterised in that the biological is a macromolecule or a group of macromolecules being an active pharmaceutical compound or an enzyme.

10. Process unit according to any one of the claims 6 to 9, characterised in that the Expanded Bed Specific Adsorption means are dense solid supports, such as mineral oxide, integrated in a chromatography column having a mineral oxide matrix with a pore volume which is less than 50% of the total volume of the highly dense matrix, and an interactive polymer network which is rooted in pores and on the surface of the mineral oxide matrix and in that said support comprises specific ligands to the biological(s) of interest to be recovered and purified.

11. Process unit according to the claim 10, characterised in that the ligand is protease-resistant, sanitisable, and has been selected to be highly specific for the biological(s) of interest.
